(19) United States
(12) Patent Application Publication
(10) Pub. No.: US 2022/0381600 A1
(43) Pub. Date: Dec. 1, 2022

(54) DEVICE AND METHOD FOR DISPENSING DROPS OF LIQUID

(71) Applicant: FAMILY SELF CARE, Paris (FR)

(72) Inventors: Brigitte THITO, Paris (FR); Christophe EVERARTS, Paris (FR)

(21) Appl. No.: 17/755,789

(22) PCT Filed: Nov. 9, 2020
(86) PCT No.: PCT/EP2020/081543

§ 371 Date: May 8, 2022

(30) Foreign Application Priority Data
Nov. 8, 2019 (FR) FR1912579

(51) Int. Cl. G01F 11/08; A61L 9/12; B05B 1/02; B05B 11/04

(52) U.S. Cl. CPC G01F 11/08; A61L 9/12; B05B 1/02; B05B 11/048; A61L 2209/111; A61L 2209/134

(57) ABSTRACT

Device (145) for dispensing drops of liquid, characterised in that it comprises: —at least one container (100) for a liquid, comprising at least one internal chamber and an upper opening, each chamber having a lower aperture and a seal sealing the upper opening, the seal having a substantially planar resiliently deformabie flexible portion, —at least one mechanical actuation means (146) configured to deform a flexible portion of a seal of at least one container, —a means (147) for selecting the volume of a drop to be dispensed from a plurality of drop volumes, a means (148) for determining the volume of liquid present in at least one container, and—a means (149) for controlling the actuation means, which means controls the movement from at least one point of the flexible portion of a container seal, as a non-constant function of the drop volume to be dispensed and the volume of liquid present in the container.

### TECHNICAL FIELD OF THE INVENTION

The invention concerns a device and a method for dispensing drops of liquid, in particular an aromatic liquid, such as essential oils or vegetable oils used in aromatherapy.

### STATE OF THE ART

A certain number of liquids such as aromatic liquids, such as essential oils for example, have the feature of degrading over time in the absence of particular precautions taken for their preservation. Document WO90/03192 describes small containers for containing and preserving such aromatic liquids. These containers are cylindrical in shape and comprise, at the bottom, an aperture for dispensing a calibrated drop of aromatic liquid and, at their upper end, a flexible elastomer cap which is elastically deformable so as to form a dropper-type device. However, such containers have the drawback that, when handling the containers prior to their manual nomadic use or prior to their placement in a quiver of a dispenser, the contained aromatic liquid comes into more or less prolonged contact with the elastomer cap, which increases the risks of degradation of the aromatic liquid prior to its manual nomadic use or in the dispenser.

Known liquid dispensing devices do not allow the dispensing of drops with differentiated volumes to be controlled.

### DISCLOSURE OF THE INVENTION

The present invention aims to remedy all or part of these drawbacks.

To this end, the present invention is aimed, according to a first aspect, at a device for dispensing drops of liquid comprising:
at least one container for a liquid comprising at least one internal chamber and an upper opening, each chamber being provided with a lower aperture, and a seal sealing the upper opening, the seal having a substantially planar elastically deformable flexible portion,
at least one mechanical actuation means configured to apply a deformation to a flexible portion of a seal of at least one container,
a means for selecting a volume of a drop to be dispensed, from a plurality of drop volumes,
a means for determining the volume of liquid present in at least one container and
a means for controlling the actuation means, which controls the movement of at least one point of the flexible portion of a container seal, according to a non-constant function of the volume of the drop to be dispensed and the volume of liquid present in the container.

Thus, the control means and the mechanical actuation means are jointly configured to effect movements of the flexible portion of at least one container according to a plurality of amplitudes and/or speeds of movement causing drops of different, preselected volumes to be dispensed.

It is noted that the controlled movement may vary in duration, speed and/or amplitude.

In embodiments, the means for controlling the actuation means controls a speed of movement of at least one point of the flexible portion of the container seal.

In embodiments, the means for controlling the actuation means controls an amplitude of movement of at least one point of the flexible portion of the container seal.

As discussed above, the magnitude and speed of deformation of the flexible portion impacts the volume of the drop dispensed. Modulation of one and/or the other allows drops of different pre-selected volumes to be produced.

In embodiments, the dispensing device comprises a temperature sensor, the means for controlling the actuation means controlling a pulse duration, speed and/or amplitude which, for a preselected drop volume, varies as a function of temperature.

Taking temperature into account compensates for thermal variations in the viscosity of the liquid.

In embodiments, the actuation means is electromechanical and the means for controlling the actuation means controls a duration, speed and/or electrical pulse of the actuation means.

It is noted that, in this case, this pulse duration impacts both the amplitude and the speed of deformation of the container seal.

In embodiments, the means for controlling the actuation means, controls a pulse duration, speed and/or amplitude which, for a preselected drop volume, is a polynomial function of degree greater than or equal to two of the volume of liquid present in the container.

The inventor has found that this polynomial function ensures the same drop volume is dispensed as the container empties and, as a result, the volume of liquid remaining in the container decreases.

In embodiments, the means for determining the volume of liquid present in at least one container comprises a liquid volume sensor.

For example, this sensor is a strain gauge in a support of the container, the strain being a linear function of the weight of liquid in the container and hence the volume of liquid in the container. According to another example, the sensor is an optical sensor that detects a meniscus formed on the surface of the liquid present in the container. According to another example, this sensor is an optical sensor that receives an amount of light reflected from the container or transmitted through the container differently depending on whether liquid is in the optical path of the light rays reaching the sensor or not. According to another example, the sensor is an electric or magnetic field sensor modulated by the presence of liquid in the container.

In embodiments, the means for determining the volume of liquid present in at least one container comprises a means for counting down, to the initial volume of liquid in the container, the volumes of drops already dispensed from the container.

These embodiments have the advantage that no sensor is required.

In embodiments, the dispensing device comprises a pressing sensor for the actuation means on the flexible portion of the seal.

By means of these arrangements, the tolerances in the positioning of the containers and/or the seals on the containers are compensated for by checking the pressing of the actuation means before the deformation motion of the flexible portion is carried out by this actuation means.

In embodiments, the actuation means comprises an electromagnet.

In embodiments, the dispensing device comprises means for receiving drops of liquid dispensed by projection into the air from a container of the dispensing device.

In embodiments, at least one container for a liquid comprises at least one internal chamber and an upper opening, each chamber being provided with a lower aperture, and a seal sealing the upper opening, the seal having a substantially planar elastically deformable flexible portion.

Throughout the present application, “substantially planar” refers to a portion which, for at least one plane, has a ratio of its surface area to the surface area of its projection onto said plane which is less than or equal to 1.5.

By means of these provisions, pressing by a single actuator, for example a user's finger or a mechanical actuator, causes the flexible portion to move a predetermined volume. This predetermined volume and the speed of movement of the flexible portion determine the calibrated volume of the drop dispensed from the lowest internal chamber of the container.

In embodiments, the resistance to deformation of the flexible portion towards the inside of the container has a decrease when the flexible portion deforms.

By means of these arrangements, when an actuator is pressed, the flexible portion deforms abruptly from one geometrical configuration to another and the predetermined volume is thus calibrated. In addition, the flexible portion, or even the entire seal, may be made of a metal that is inert to the liquid to be dispensed.

In embodiments, the seal comprises a cap which can be removed from the opening, which cap carries the flexible portion.

In embodiments, the container comprises a main chamber comprising an amount of liquid and comprising a lower liquid dispensing aperture, and a secondary chamber positioned between the seal and the main chamber, the secondary chamber being separated from the main chamber by a tight partition wall provided with the lower aperture of the secondary chamber, which places the main chamber and the secondary chamber in fluid communication.

In embodiments, the open surface area of the lower aperture of the secondary chamber is smaller than the open surface area of the dispensing aperture of the main chamber.

In embodiments, the inner volume of the secondary chamber is at least three times smaller than the inner volume of the main chamber.

Advantageously, but optionally, the container has at least one of the following technical characteristics:
the partition wall is planar;
the partition wall is generally frustoconical in shape, the apex of which extends into the main chamber and carries the lower aperture of the secondary chamber;
the container has a rotational symmetry, the container being generally cylindrical or conical in shape, for example;
the dispensing aperture is inclined with respect to a longitudinal axis of the container;

the liquid contained in the main chamber is one of an essential oil and a vegetable oil; and
the container is made partly of glass.

In embodiments, the device comprises a carousel comprising a structure for receiving and supporting a plurality of containers.

In embodiments, the carousel comprises mechanical elements (such as springs or clips) for compensating for tolerances of the liquid containers.

In embodiments, the device further comprises a removable aperture seal positioned below the apertures and having a rotatable connection, and comprising as many supports for stoppers as there are containers, rotation of the aperture seal in one direction positioning the stoppers in contact with the dispensing apertures, rotation in the other direction moving these stoppers away from these apertures. By means of this seal, liquid leakage is thereby avoided, for example, during pressure increases (due to temperature or altitude increases) or accelerations (due to shocks/vibrations or motions). This seal also avoids evaporation/degradation of liquid between uses of the device.

The stoppers of the seal are designed in such a way that their shape (dome or cylinder) and their material (in particular elastomer), hermetically seal the apertures of the containers.

In some embodiments, the device also comprises a position sensor for the aperture seal, the drop ejection control means being configured to control drop ejection only when the stoppers are moved away from the apertures of the containers.

Drop ejection control is thus inhibited when the stopper seals the lower aperture of each container.

In embodiments, the sealing device is manually controlled by the end user, by opening and closing the seal when a dedicated visual or audible signal is provided by the device.

In embodiments, the seal device is semi-automatically controlled by the end user, by opening the seal when a dedicated visual or audible signal is provided by the device; the device closing the seal automatically (mechanically or electromechanically).

In embodiments, the seal device is automatically controlled with the seal opening and closing automatically as required.

For example, electromechanical means are implemented for these semi-automatic or automatic controls.

The various particular characteristics of the dispensing device as described above and in the description and the particular characteristics of the container as described above and in the description are for being combined with each other to realize dispensing devices subjects of the invention.

According to a second aspect, the present invention relates to a method for dispensing drops of liquid from at least one container for a liquid comprising at least one internal chamber and an upper opening, each chamber being provided with a lower aperture, and with a seal sealing the upper opening, the seal having a substantially planar elastically deformable flexible portion,
a step of selecting a volume of a drop to be dispensed, from a plurality of drop volumes,
a step of determining the volume of liquid present in at least one container and
a step of controlling at least one mechanical actuation means configured to apply a deformation to a flexible portion of a seal of at least one container, the movement of at least one point of the flexible portion of a container seal, according to a non-constant function of the volume of the drop to be dispensed and the volume of liquid present in the container.

As the advantages, purposes and particular characteristics of this method are similar to those of the device which is the subject of the present invention, they are not reminded here.

### BRIEF DESCRIPTION OF THE FIGURES

Further advantages, purposes and particular characteristics of the invention will be apparent from the following non-limiting description of at least one particular embodiment of the device which is the subject of the present invention, with reference to the appended drawings, in which:

FIG. 1 is a longitudinal cross section view of a first embodiment of a liquid container;

FIG. 2 is a longitudinal cross section view of a second embodiment of a liquid container;

FIG. 3a is a first longitudinal cross section view of the container of FIG. 2 illustrating a use of the container in a liquid drop dispensing device which is the subject of the invention;

FIG. 3b is a second longitudinal cross section view of the container of FIG. 2 illustrating a use of the container in a liquid drop dispensing device which is the subject of the invention;

FIG. 3c is a third longitudinal cross section view of the container of FIG. 2 illustrating a use of the container in a liquid drop dispensing device which is the subject of the invention;

FIG. 4 represents, in a partial side view, a third embodiment of a liquid container fitted with a cap below an actuation means;

FIG. 5 represents, in a partial side view, a fourth embodiment of a liquid container;

FIG. 6 represents, in a side view, a container cap;

FIG. 7 represents, in a cross section view, a container cap;

FIG. 8 represents, in a top view, a container cap;

FIG. 9 represents, in a top view, an alternative of the container cap the flexible portion of which bulges towards the actuator;

FIG. 10 represents, in a side view, the alternative of the cap illustrated in FIG. 9;

FIG. 11 represents, in perspective, a carousel of a liquid drop dispensing device which is the subject of the invention;

FIG. 12 represents, in perspective and in a partial view, a liquid drop dispensing device, which is the subject of the invention;

FIG. 13 represents, as a logic diagram, the operating steps of a liquid drop dispensing device, which is the subject of the invention, and

FIG. 14 represents, in perspective, a seal for a carousel illustrated in FIG. 11.

### DESCRIPTION OF THE EMBODIMENTS

For the sake of clarity, identical or similar elements are marked with identical reference signs throughout the figures.

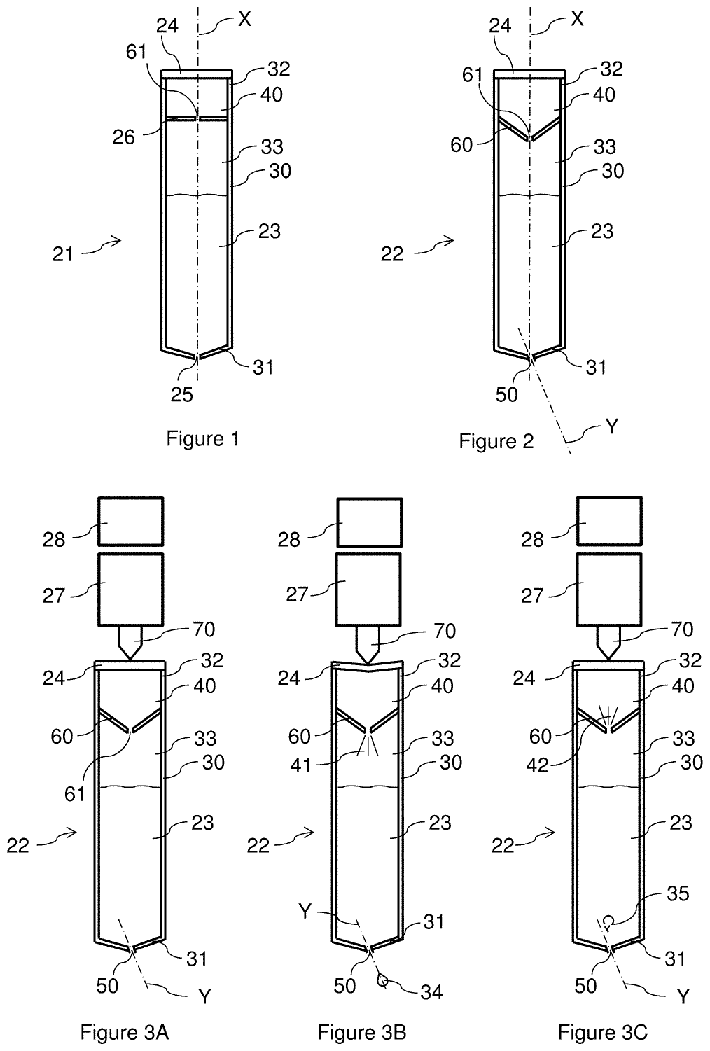

With reference to FIG. 1, a first embodiment of a liquid container 21 will be described in more detail. The container 21 contains, or is for containing, a liquid 23, such as, for example, without being limiting thereto, an essential oil or a vegetable oil, or a cosmetic, therapeutic or food liquid. The container 21 is generally cylindrical in shape and elongated along a longitudinal axis X. Here, the container 21 is cylindrical in shape and has a rotational symmetry along the longitudinal axis X. The container 21 has a lower end 31 and an upper opening or end 32. At its lower end 31, the container 21 has a dispensing aperture 25 arranged such that, when the container 21 is being used, the dispensing aperture 25 delivers a calibrated drop 34 (see FIG. 3b) of liquid 23. Here, the dispensing aperture 25 is coaxial with the longitudinal axis X. When a drop of liquid 23 is being dispensed, the axis X is substantially vertical as illustrated in FIG. 1.

Extending from the lower end 31 along the longitudinal axis X towards the upper opening 32, the container 21 comprises a first or main chamber 33, laterally delimited by a wall 30, and comprising, or for receiving, an amount of liquid 23. The main chamber 33 is delimited at the top by a partition wall 26 extending, here, across the container 21. Furthermore, the main chamber 33 is in fluid connection with the dispensing aperture 25.

Between the partition wall 26 and the upper opening 32, the container 21 comprises a second or secondary chamber 40 delimited at the bottom by the partition wall 26, and laterally by the wall 30. The secondary chamber 40 is in fluid communication with the main chamber 33 via fluid connection means, here comprising a calibrated aperture 61 passing through the partition wall 26. These fluid connection means are at least tight against the liquid 23, in particular during any handling of the container 21. This means that if the container 21 is turned upside down or laid down, the liquid 23 contained in the main chamber 33 cannot flow or leak into the secondary chamber 40 through the fluid connection means, here the aperture 61 calibrated to this end. The secondary chamber 40 comprises air or any other gas. It should be noted that the fluid connection means, in this case the calibrated aperture 61, are not tight against air, or any other gas contained in the secondary chamber 40, the latter being able to flow from one chamber to the other.

In embodiments, the surface area of the smallest sectional area of the calibrated aperture 61 is less than or equal to the surface area of the smallest sectional area of the calibrated aperture 25. Even more preferably, the surface area of the smallest sectional area of the calibrated aperture 61 is less than or equal to a quarter of the surface area of the smallest sectional area of the calibrated aperture 25. For example, the calibrated apertures 25 and 61 are cylindrical with a circular directrix and have diameters of 1 mm and 0.5 mm respectively. Thus, the surface tension forces prevent the liquid 23, especially if it is an essential oil or vegetable oil, from passing through the aperture 61, especially if the internal wall 26 is made of glass.

In embodiments, the surface area of the smallest sectional area of the calibrated aperture 61 is of the same order of magnitude in size as that of the calibrated aperture 25. In embodiments, the surface area of the smallest sectional area of the calibrated aperture 61 is larger than the surface area of the smallest sectional area of the outlet aperture 25.

It is noted that the person skilled in fluid mechanics knows, depending on the type and viscosity of the liquid 23 and the material constituting the internal wall 26, how to determine the geometry of the aperture 61 to prevent the liquid 23 from flowing into the secondary chamber 40 when the container 21 is laid down or turned upside down and, preferably, also when the container 21 is shaken or agitated when being transported, installed in a carousel or in a dispensing device and when used.

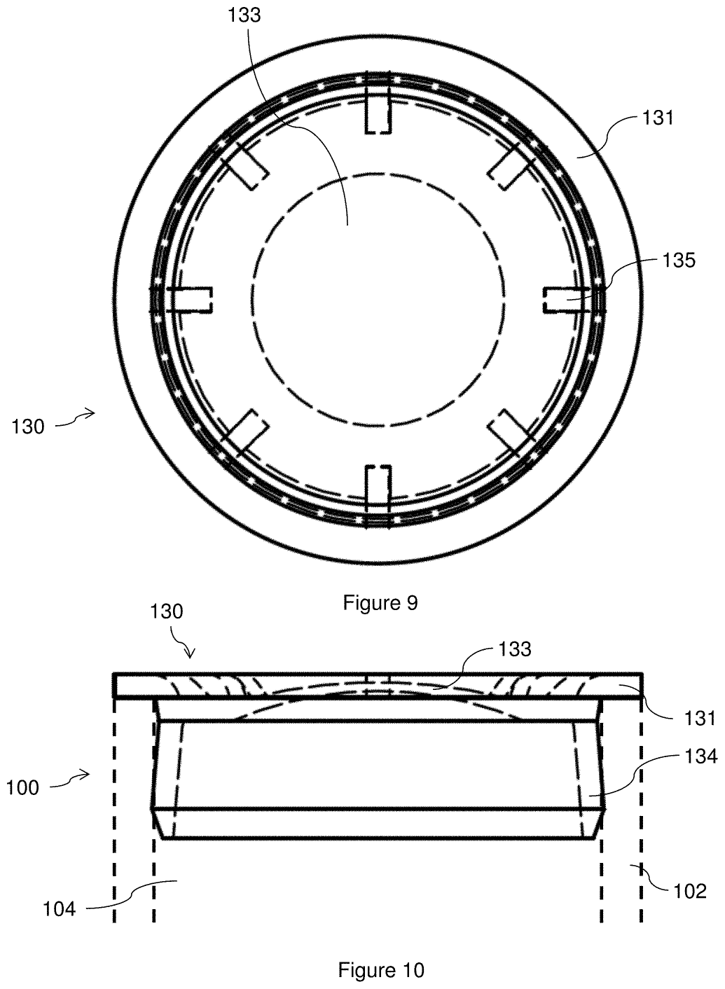

In addition, the air within the secondary chamber applies pressure to the liquid which retains the liquid in the first chamber. The smaller the volume of the secondary chamber, the greater the pressure increase within the secondary chamber when a meniscus is formed by the liquid at the aperture 61. In addition, when the elastically deformable flexible portion bulges towards the outside of the container (see FIGS. 9 and 10), the resistance to deformation of this part is higher for a deformation towards the outside of the container than for a deformation towards the inside of the container, which again favours the retention of the liquid in the first chamber.

The container 21 also has, at its upper opening 32, a seal or membrane 24 which closes an opening of the container 21 and its secondary chamber 40. The seal or membrane 24 is flexible and elastically deformable and integrated into a closing cap of the secondary chamber 40. The membrane 24 is further arranged to form pumping means, in the manner of a dropper system, allowing a release of a calibrated drop 34 of liquid 23, as will be explained later in relation to FIGS. 3a to 3c illustrating a second embodiment of a liquid container 22.

With reference now to FIG. 2, the second embodiment 22 of the liquid container is described. However, only the differences between it and the previously described container 21 will be described. The container 22 differs firstly in that the dispensing aperture 50 has a longitudinal axis Y which is inclined at an angle α to the longitudinal axis X of the container 22. This angle, preferably between 0° and 45°, allows for a deflected release of the calibrated drop 34 of liquid 23 when the container 22 is being used.

The container 22 also differs from the container 21 in that the partition wall 60 is frustoconical in shape, with the calibrated aperture 61 being, for example, arranged at the apex of the frustoconical shape. Alternatively, the calibrated aperture 61 is formed at a frustoconical wall of the partition wall 60. The frustoconical shape of the partition wall 60 extends, in FIG. 2, into the main chamber 33.

In an alternative embodiment, the liquid container has only one of the above two differences from the first embodiment of the container 21.

The entire container, 21 or 22, is made, for example, of glass, preferably opaque, apart from the membrane 24 which is made of elastomer.

With reference to FIGS. 3a to 3c, a use of the container, 21 or 22, previously described within a liquid drop dispensing device, or “dispenser” 28 subject of the invention, will now be described.

The dispenser 28 comprises a receptacle for one or more containers, 21 or 22, previously described. It should be noted that a liquid carousel may be used in the receptacle. To this end, the carousel comprises a structure for receiving and supporting a plurality of containers 21 or 22 just like a quiver. The dispenser 28 comprises at least one actuation means 27 arranged to press on the membrane 24 when dispensing the calibrated drop 34 of liquid 23. Here the actuation means comprises an electromagnet 27 maneuvering a pressing finger 70 coming into contact with the membrane 24. In other embodiments, the actuation means comprises a piezoelectric crystal. The dispenser may have as many actuation means 27 as there are containers 21 or 22 within the device 28.

FIG. 3a illustrates a rest position between the actuation means 27 and the container 21 or 22. In this position, the finger 70 is in contact without pressing on the membrane 24 which is in a rest position. In an alternative embodiment, the finger 70 is not in contact with the membrane and has a free end positioned at a distance from and facing said membrane 24.

Then, as illustrated in FIG. 3b, for dispensing the calibrated drop 34 of liquid 23, the finger 70 presses on the membrane 24 deforming it towards the inside of the secondary chamber 40, creating within the secondary chamber 40 an increase in the internal pressure of the gas present. As a result, a flow of pressurised gas 41 passes through the calibrated aperture 61 of the secondary chamber 40 towards the main chamber 33, creating an overpressure within the main chamber 33. This overpressure thus created within the main chamber 33 causes the calibrated drop 34 of liquid 23 to be dispensed by expelling the liquid 23 through the dispensing aperture 25, 50 from the main chamber 33.

Then, in FIG. 3C, the finger 70 releases its pressing on the membrane 24 which, due to its elasticity, returns to its rest position, creating a negative pressure within the secondary chamber 40 which in turn creates a flow of air 42 through the calibrated aperture 61 from the main chamber 33 to the secondary chamber. A negative pressure is then created within the main chamber 33, which results in the introduction of an air bubble 35 through the dispensing aperture 25, 50 into the main chamber 33. It is clear from the above that such a structure of the container 21 or 22 avoids any contact between the liquid 23 and the membrane 24.

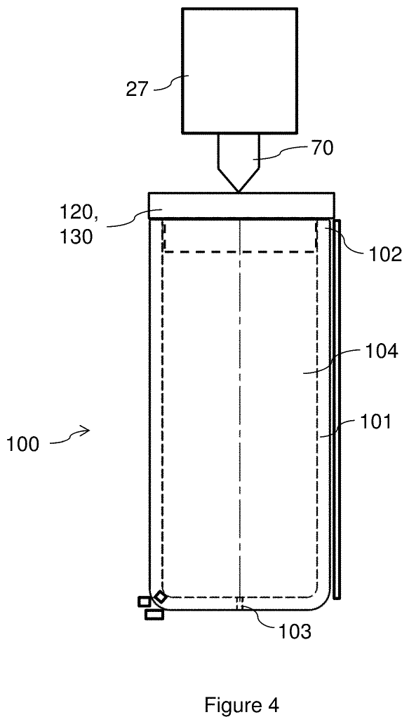

FIG. 4 shows a portion, without a seal, of a third embodiment 100 of a liquid container. This container 100 has an external wall 101, for example made of glass, in the shape of a truncated cylinder ending, in its top, in an opening 102 and, in its bottom, in a planar external wall, perpendicular to the generatrix of the cylinder and comprising a dispensing aperture 103. The container 100 comprises only a single chamber 104. Alternatively, the container has another general shape with rotational symmetry, for example conical. Alternatively, the dispensing aperture 103 is inclined with respect to a longitudinal axis of the container 100.

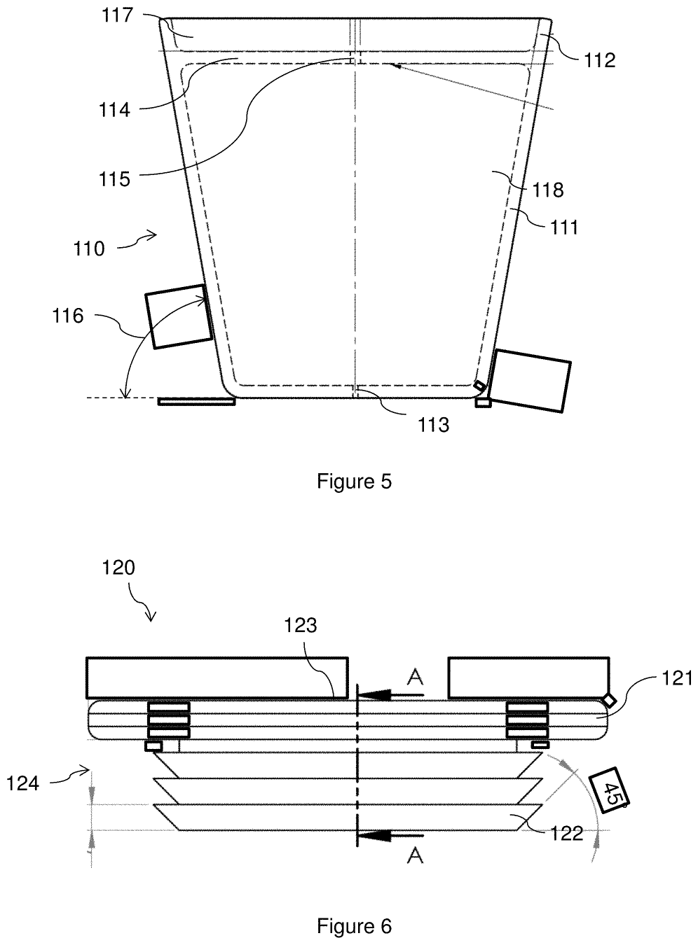

FIG. 5 shows a portion, without a seal, of a fourth embodiment 110 of a liquid container. This container 110 has an external wall 111, for example made of glass, in the shape of a truncated cone ending, in its top, in an opening 112 and, in its bottom, in a planar external wall, perpendicular to the axis of the cone and comprising a dispensing aperture 113. The angle 116 defining the cone is, for example, 80°. In this embodiment, an internal wall 114 separates an upper chamber 117, called the secondary chamber, comprising the opening 112 and a lower chamber 118, called the main chamber. An aperture 115 passes through the internal wall 114. Alternatively, the container 110 has another general shape with rotational symmetry, for example cylindrical. Alternatively, the dispensing aperture 113 is inclined with respect to a longitudinal axis of the container 110.

As set out in relation to FIGS. 1 to 3c, preferably the aperture 115 does not allow the passage of liquid from the main chamber 118 to the secondary chamber 117.

Also in the embodiments of FIGS. 4 and 5, a flexible portion seals the upper opening, 102 or 112, of the container. This flexible portion can either be incorporated directly into the container as a single piece, or be in the form of a foil lid integral with the outer wall, 101 or 111, of the container, or in the form of a cap closing the top of the container, or else an independent membrane fixed by means of an external device (by pinching for example). The pumping action is achieved by deforming the flexible portion of the seal. This action can be carried out in several ways. When the deformation induces a decrease in the volume in the container 100 or 110, this creates an overpressure in the container 100 or 110 and liquid comes out through the lower dispensing aperture, 103 or 113. On the contrary, when the deformation induces an increase in volume in the container 100 or 110, this creates a negative pressure and air enters through the lower dispensing aperture, 103 or 113.

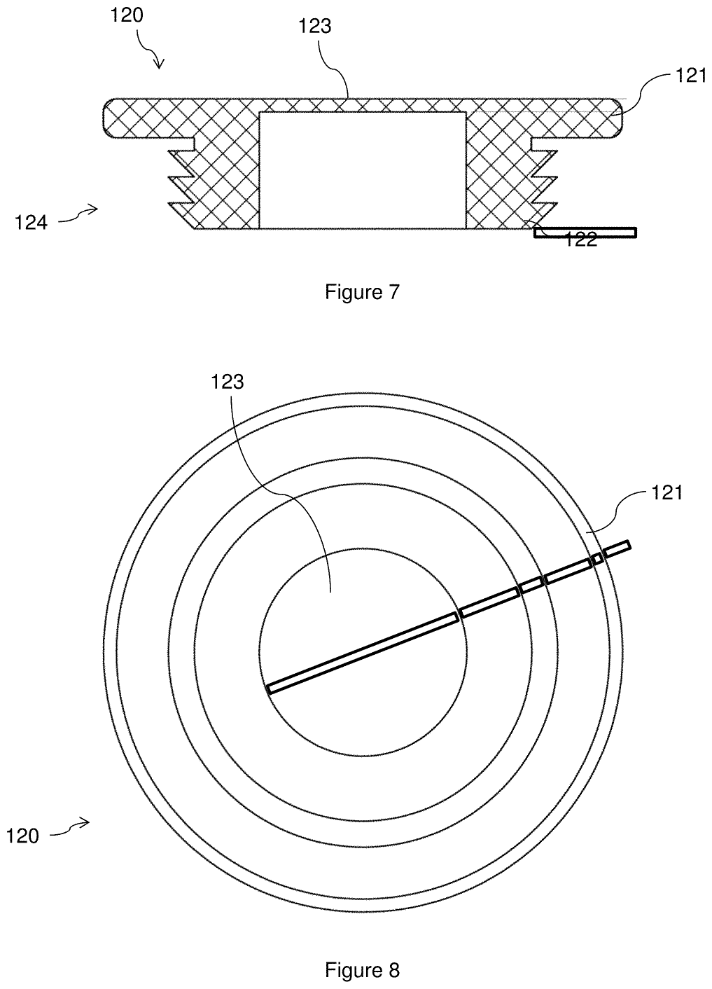

A cap 120 for a cylindrical container with a circular directrix, for example container 100, is shown in FIGS. 6 to 8. The cap 120 comprises a circular base 121 whose surface area is larger than that of the opening 102. This base 121 carries, in its central portion, a substantially planar elastically deformable flexible portion 123. This base 121 also carries an elongation 124 provided with circular teeth of external dimensions greater than the surface area of the opening 102 and of internal dimensions less than the surface area of the opening 102. By pressing the base 121 towards the teeth 122, these teeth 122 tightly seal the opening 102.

A possible method of operation may be as follows: an actuator applies pressure to the top of the flexible portion which deforms. This causes a drop to be expelled. The actuator then releases the pressure and an air bubble enters the container. The actuation of this flexible portion can be carried out in different ways: either in an automated way in a machine using a pneumatic, hydraulic, electromagnetic or electric actuator, or in a manual way by pressing by hand directly on the flexible portion or through a mechanism. In this second case, the container is used in a nomadic way.

The calibration of the drops is done through several parameters. Indeed, the volume of the drop depends on the size of the dispensing aperture, the viscosity of the liquid, the volume moved by the deformation of the flexible portion and the speed at which this flexible portion deforms. The volume of deformation of the flexible portion depends on the stiffness of the flexible portion as well as the force exerted on the flexible portion and the speed of motion. This force can be either continuous, in which case the volume of the drop is equal to the volume variation induced by the flexible deformation, or impulsive. In the latter case, a drop is ejected, its volume depending on the size of the dispensing aperture, the volume of deformation and the duration, speed and/or amplitude of the pulse.

The seal is made from materials compatible with essential oils, for example materials based on Polypropylene (“PP”) and/or based on a polymer softer than Polypropylene, for example Vistamax (registered trademark), Purell (registered trademark) or Lupolen (registered trademark). For example, the material of which the flexible portion of the cap is made comprises at least 25% Polypropylene. According to another example, the material of which the flexible portion of the cap is made comprises more than half Polypropylene and less than half Vistamax. The geometry of the seal is based on tests to optimise the degree of deformability. The geometry represented in FIGS. 9 and 10, when scaled for a cylindrical or conical container with an opening of 15-35 millimetres internal diameter, is an example of a geometry that allows calibrated drops to be obtained. The thickness of the flexible membrane is, for example, less than one millimetre and, more particularly, less than 0.5 millimetres in its thinnest part. This thickness is, for example, 0.3 mm.

As can be understood upon reading the preceding description, the liquid container comprises at least one internal chamber and an upper opening, each chamber being provided with a lower aperture, and a seal sealing the upper opening, the seal having a substantially planar, elastically deformable flexible portion. It is reminded that “substantially planar” refers to a portion which, for at least one plane, has a ratio of its surface area to the surface area of its projection onto said plane which is less than or equal to 1.5. Thus, the pressing of a single actuator, for example a user's finger or a mechanical actuator, causes the flexible portion to move a predetermined volume. This predetermined volume and the speed of movement of the flexible portion determine the calibrated volume of the drop dispensed from the lowest internal chamber of the container.

In FIGS. 9 and 10, a top view and a side view of a cap 130 are shown. In FIG. 10, the opening 102 of the container 10 is shown partially and in dotted lines to indicate the geometric relationship between the cap 130 and the opening 102 of the container 101. This cap 130 comprises a circular base 131 whose surface area is greater than that of the opening 102. This base 131 carries, in its central part, a substantially planar elastically deformable flexible portion 133. In alternatives such as the one represented, radial ribs 135 act as a stiffener for the portion of the cap 130 which surrounds the flexible central portion 133, in order to limit the unwanted risks of deformation. In other alternatives, the cap 130 does not have radial ribs. The base 131 also has an elongation 134 configured to be secured by tight fit into the opening 102 of the container 100.

In the embodiment of a cap 130 illustrated in FIGS. 9 and 10, the central flexible portion 133 bulges towards the outside of the container. Thus, the resistance to deformation of the flexible portion 133 towards the inside of the container exhibits a decrease when this flexible portion deforms. As a result, when an actuator is pressed, the flexible portion deforms abruptly from one geometrical configuration to another and the predetermined volume is thus calibrated. In addition, the flexible portion, or even the entire seal 130, may be made of a metal inert to the liquid to be dispensed.

Bulging a portion of the surface of the seal towards the outside, for example in the form of a truncated sphere, has the effect that the resistance to depression is, as a function of the distance covered by the actuation means from the first pressing on this membrane:
first increasing, as long as the whole bulged portion is in compression,
and then decreasing, when this portion buckles, that is forms a fold.

For example, the radius of curvature of the external surface (top in FIG. 10) is between 16 and 20 millimetres, preferably between 17.5 and 18.5 millimetres, and the radius of curvature of the internal surface (bottom in FIG. 10) is between 11 and 15 millimetres, preferably between 12 and 13.5 millimetres.

In the embodiments illustrated in FIGS. 1 to 3b and 5, the liquid container comprises a main chamber having an amount of liquid and having a lower liquid dispensing aperture, and a secondary chamber positioned between the seal and the main chamber, the secondary chamber being separated from the main chamber by a tight partition wall provided with the lower aperture of the secondary chamber, which places the main chamber and the secondary chamber in fluid communication. The open surface area of the lower aperture of the secondary chamber is smaller than the open surface area of the dispensing aperture of the main chamber. The inner volume of the secondary chamber is smaller, preferably at least three times smaller than the inner volume of the main chamber, more preferably at least five times smaller and even more preferably nine times smaller.

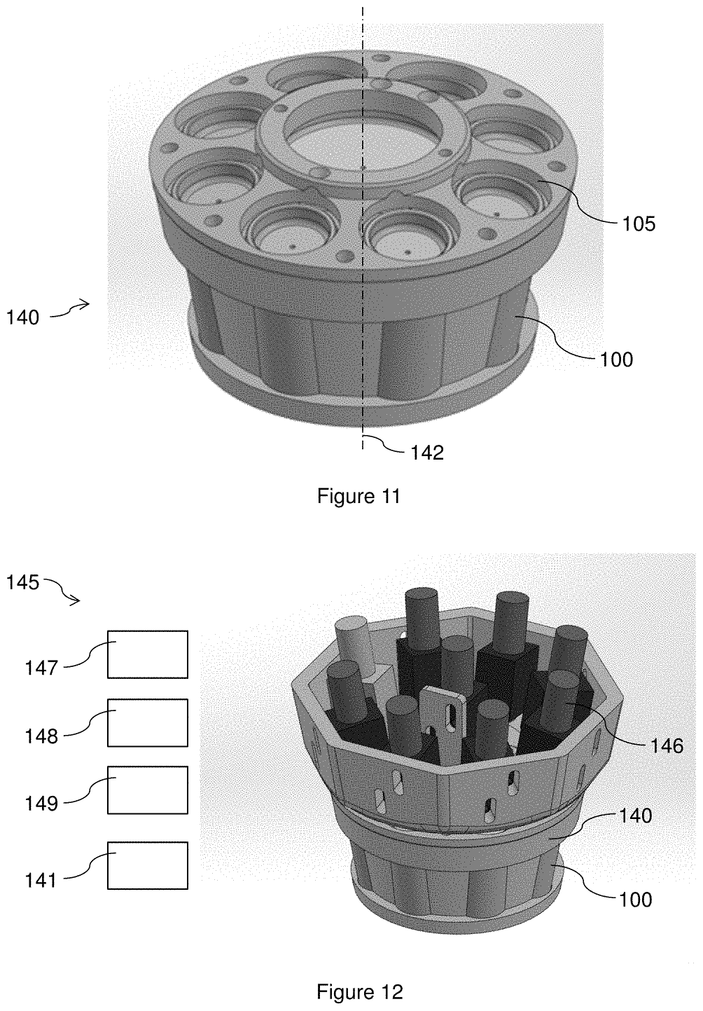

FIG. 11 shows a carousel 140 comprising a plurality of supports each configured to removably receive a liquid container, for example a container 100. In embodiments, the carousel 140 comprises mechanical elements, for example springs or clips, which allow the mechanical tolerances of the liquid containers to be compensated for.

FIG. 12 shows a portion of a liquid drop dispensing device 145 comprising a carousel 140 illustrated in FIG. 11 and at least one actuation means 146 configured to press on a flexible portion of a seal of at least one container.

With respect to the carousel 140 and the drop dispensing device 145, reference will be made to the international application PCT/FR2018/053065, incorporated herein by reference, filed on 30 Nov. 2018 with the national institute of industrial property.

Preferably, the mechanical actuation means comprises a control means configuring it to perform movements of the flexible portion of at least one container according to a plurality of amplitudes and/or speeds of movement. For example, in the case of an electromagnet actuation means, a time modulation of the current applied to the electromagnet and/or a modulation of the duration of application of a constant current temporally modulates the force exerted by the electromagnet and thus temporally modulates, on the one hand, the speed and, on the other hand, the amplitude of its movement and thus the amplitude of the deformation of the flexible portion of the container seal located facing this electromagnet. The amplitude and the speed of deformation of the flexible portion impact the volume of the dispensed drop, the modulation of one and/or the other making it possible to produce drops of different calibrated volumes.

In embodiments, the dispensing device 145 comprises a pressing sensor (not represented) for the actuation means on the flexible portion of the seal. This sensor is, for example, a sensor of the voltage and/or the intensity of the electric current passing through a coil of an electromagnet, during a slow descent of the actuation means: when the voltage and/or the electric intensity suddenly increases, the actuation means presses on the flexible portion of the seal. This sensor can also be a contact sensor with a switch positioned at the end of the actuation means or an optical sensor, for example. In this way, the tolerances in the positioning of the containers and/or the seals on the containers are compensated for by checking the pressing of the actuation means before the deformation movement of the flexible portion is carried out by this actuation means.

What is described below concerns both the containers as shown in relation to FIGS. 1 to 11 and any other container having a deformable portion on which an actuation means can press.

The dispensing device 145 also comprises:
a means 147 for selecting a volume of a drop to be dispensed, from a plurality of drop volumes,
a means 148 for determining the volume of liquid present in at least one container and
a means 149 for controlling at least one actuator means 146, which controls the movement of at least one point on the flexible portion of a container seal, according to a non-constant function of the volume of the drop to be dispensed and the volume of liquid present in the container.

Thus, the control means 149 and the mechanical actuation means 146 are jointly configured to effect movements of the flexible portion of at least one container according to a plurality of amplitudes and/or speeds of movement causing drops of different, preselected volumes to be dispensed.

In embodiments, the means 149 for controlling the actuation means 146 controls a speed of movement of the at least one point of the flexible portion of the container seal.

In embodiments, the means 149 for controlling the actuation means 146 controls an amplitude of movement of at least one point of the flexible portion of the container seal.

As discussed above, the magnitude and speed of deformation of the flexible portion impact the volume of the drop dispensed. Modulation of one and/or the other allows drops of different pre-selected volumes to be produced.

In embodiments, the dispensing device 145 comprises a temperature sensor 141, the means for controlling the actuation means controlling a pulse duration, speed and/or amplitude which, for a preselected drop volume, varies as a function of the sensed temperature. Taking temperature into account allows compensation for thermal variations in the viscosity of the liquid present in the container. In embodiments, the actuation means 146 is electromechanical, for example an electromagnet, and the means 149 for controlling the actuation means 146 controls a duration, speed and/or amplitude of an electrical pulse of the actuation means 146. It is noted that, in the case of an electromagnetic actuation means with constant electrical current between and during pulses, this pulse duration impacts both the amplitude and the speed of deformation of the container seal.

In embodiments, the means 149 for controlling the actuation means 146 controls a pulse duration, speed and/or amplitude which, for a preselected drop volume, is a polynomial function of degree greater than or equal to two of the volume of liquid present in the container. The inventor has found that this polynomial function ensures the same drop volume is dispensed as the container empties and, as a result, the volume of liquid remaining in the container decreases. An example of a second degree polynomial function is set out below.

In embodiments, the means 148 for determining the volume of liquid present in at least one container comprises a liquid volume sensor.

For example, this sensor is a strain gauge in a support of the container, the strain being a linear function of the weight of liquid in the container and thus of the volume of liquid in the container. According to another example, the sensor is an optical sensor that detects a meniscus formed on the surface of the liquid present in the container. According to another example, this sensor is an optical sensor that receives an amount of light reflected from the container or transmitted through the container differently depending on whether liquid is in the optical path of the light rays reaching the sensor or not. According to another example, the sensor is a sensor of electric or magnetic field modulated by the presence of liquid in the container.

In embodiments, the means 148 for determining the volume of liquid present in at least one container comprises a means for counting down, to the initial volume of liquid in the container, the volumes of drops already dispensed from the container. Thus, for each drop ejected from a container, the volume determining means 148 subtracts, from the volume of liquid present in the container prior to the ejection, the preselected volume of the ejected drop to obtain the new volume of liquid present in the container after the ejection. These embodiments have the advantage that no sensor is required.

In embodiments, the dispensing device 145 comprises a pressing sensor for the actuation means on the flexible portion of the seal, as discussed above. Thus, tolerances in the positioning of the containers and/or the seals on the containers are compensated for by checking the pressing of the actuation means before the actuation means performs the deformation motion of the flexible portion.

In embodiments, the dispensing device 145 comprises means for receiving drops of liquid dispensed by projection into the air from containers of the dispensing device. Since, preferably, the containers are inclined and their longitudinal axes are oriented towards a point of convergence, these receiving means are placed around this point of convergence.

Further details of a preferred embodiment of the present invention are given below.

An electromechanical device, such as an electromagnet, activates a flexible membrane, made of a deformable material, which tightly closes a container, or “vial”, containing the essential oil, by depressing the membrane. This vial has a small aperture Of at the bottom through which the drop of essential oil can pass.

The depression of the flexible membrane causes an overpressure in the vial which allows a drop of essential oil to be ejected through the small, for example circular, aperture. This depression is related to the impact force Ft of the rod of the electromagnet to deform the membrane.

This electromagnet operates with a supply voltage, an axial stroke of the membrane depressing rod and the rod pulse time.

The electromagnet is characterised by a supply voltage U, an axial stroke of the membrane depressing rod Ct and a rod pulse time Ti.

Typically, Ti is between 20 ms and 100 ms.

The membrane is characterised by its material, thickness, shape and dimensions. The material has a hardness Dm/Young's modulus Em compatible with the desired operation under the effect of the electromagnet's depression. The membrane ensures a perfect tightness within the vial to prevent air from escaping during depression and therefore a pressure drop preventing or limiting the ejection of the drop.

The material of the membrane has properties of resistance to the chemical agents contained in the essential oils. For example, LDPE (acronym for low density polyethylene). The material is inert with respect to food contact so as not to introduce external agents when in contact or proximity with the essential oil.

The material thickness epm allows a maximum depression force Ft of 5 N, to compress the inner volume and make a drop come out.

The shape of the membrane allows both ease of depression and repeatability of the volume depressed to ensure sufficient and repeatable overpressure in the vial. This membrane allows for easy and repeatable depression with the finger for nomadic applications. A spherical, blister-shaped bulged membrane is used to meet these requirements. It allows the internal pressure of the vial to be increased during depression with the rod, to a greater extent than a planar membrane, because it has a reserve of volume in the bulge of its shape. In addition, it allows for better metering of this pressure with this inner volume of the blister shape.

The stroke of the electromagnet is, for example, between 1 and 1.5 mm.

The invention aims to control a variable drop volume Vg, this drop being ejected at the vial outlet, to ensure a dispense of essential oils with predetermined proportions. This drop size Vg is related to the following parameters
Impact force/membrane deformation Fm
Pulse time Ti
Viscosity of the liquid Vi
Volume of essential oil liquid in the vial Vhe
Diameter of the outlet aperture Of
Material of the vial Mf (related to the wettability and surface tension of the liquid).

There is therefore a complex relationship Vg=f(Fm, Ti, Vi, Vhe, Of, Mf).

To select the size and therefore the volume of the drop, the drop dispensing device controls the impact force Fm on the membrane, which is related to the force Ft of the rod. This force Fm is related to the following parameters
Electromagnet supply voltage U
Pulse time Ti
Electromagnet—membrane distance related to Ct
Membrane flexibility Dm and Em
Membrane thickness epm
Shape of the membrane
Diameter of the blister.

There is therefore a complex relationship Fm=f(U, Ti, Ct, Dm, Em, epm, blister shape)

The parameters of the above equations have influences on the final result: On the impact initial energy/force:
The shorter Ti is, the greater Fm is
The higher U is, the higher Fm is

On the ease with which the membrane can be depressed:
The lower Em is, the lower Fm will be to depress (and vice versa)
The lower epm is, the lower Fm will be to depress
The larger the blister shape diameter is, the lower Fm will be to depress
The larger the radius of curvature of the blister is, the greater Fm will be to depress

The inventor has found a family of equations defining the size and volume of an ejected drop.

There are three types of parameters that influence the volume of an ejected drop:
parameters that are fixed in a material way by calibration: voltages, dimensions and mechanical tolerances, etc. These parameters have been defined at the design stage and have been imposed on the equipment,
parameters that are variable and defined by the environment: temperature, ageing, etc. These parameters have to be measurable in order to take them into account in the calculations (need for sensors),
parameters that are variable and defined by the machine: pulse duration, pulse speed and/or pulse amplitude, oil used, etc.

For given fixed parameters, the inventor has found a relationship between the pulse duration, for a pre-selected drop volume, and certain measurable or determinable parameters:

t=a*V²+b*V+c*T+d

where:

t=the pulse duration that has to be applied to obtain a drop of the targeted volume

V=the volume of liquid remaining in the vial

T=the room temperature.

For the implementation of the control means of the electromagnet (particular actuation means), the control means stores values of the constants a, b, c and d for each liquid present in a container.

In alternative embodiments, the control means implements a polynomial function that does not take the temperature into account, the temperature being considered as being the room temperature of 20° C. In alternatives, the control means implements a polynomial function of degree greater than two.

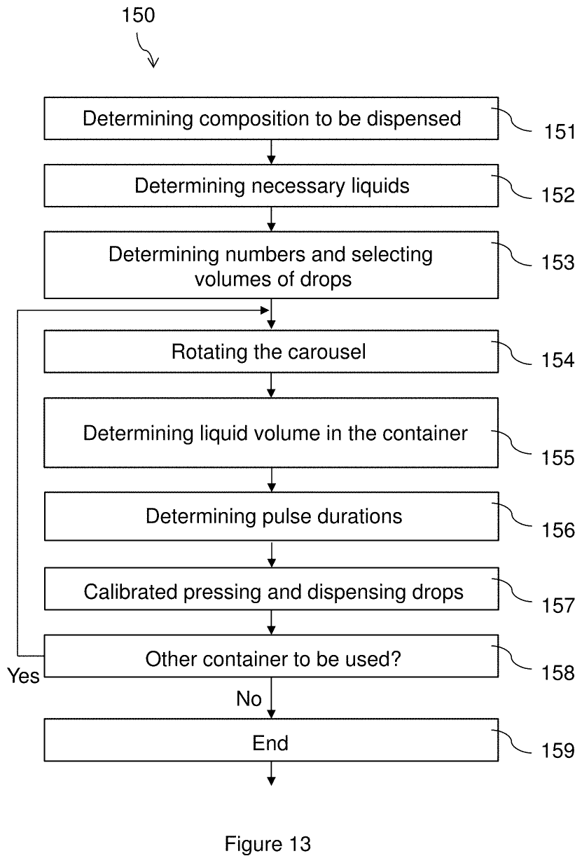

In the method 150 illustrated in FIG. 13, in a step 151, the composition of liquids to be dispensed is determined. For example, this determination is carried out according to a user profile and the needs of this user. In a step 152, the containers of liquids to be implemented are determined. These containers correspond to the liquids entering the composition determined during step 151. In a step 153, the number and volume of calibrated drops to be dispensed by each selected container are determined, depending on the composition to be obtained. In a step 154, in the case where only one actuator is used, the carousel is rotated to place a first selected container facing this actuator.

In a step 155, the volume of liquid present in the container is determined by measurement or estimation. In a step 156, depending on the volumes of calibrated drops to be dispensed, the amplitude and speed of the motion of the actuator for the liquid in question is determined. As set out above, both the amplitude and speed of motion influence the volume of drops dispensed.

In the case where the actuator means is electromechanical with a constant current between and during pulses, the electrical pulse durations corresponding to the drops to be dispensed from the liquid present in the container are determined. For example, the polynomial relationship described above is implemented to determine this electrical pulse duration.

In a step 157, the actuator presses on the flexible portion of the container seal with the determined amplitude of motion and speed of motion, for example with the electrical pulse duration determined in step 153. The number of calibrated drops and their volumes determined in step 153 are thus dispensed. In a step 158, it is determined whether there is still at least one liquid to be dispensed. If so, the method returns to step 154 to present a corresponding container facing the actuator. If not, the method is terminated at step 159.

It is noted that, in the case where the carousel is provided with as many actuators as there are containers, steps 154 and 158 are omitted and steps 157 relating to the different liquids and containers can be carried out simultaneously.

Thus, in embodiments, the method for dispensing drops of liquid from at least one container comprises:
a step of selecting a volume of a drop to be dispensed, from a plurality of drop volumes,
a step of determining the volume of liquid present in at least one container and
a step of controlling at least one mechanical actuation means configured to apply a deformation to a flexible portion of a seal of at least one container, the movement of at least one point of the flexible portion of a container seal, according to a non-constant function of the volume of the drop to be dispensed and the volume of liquid present in the container.

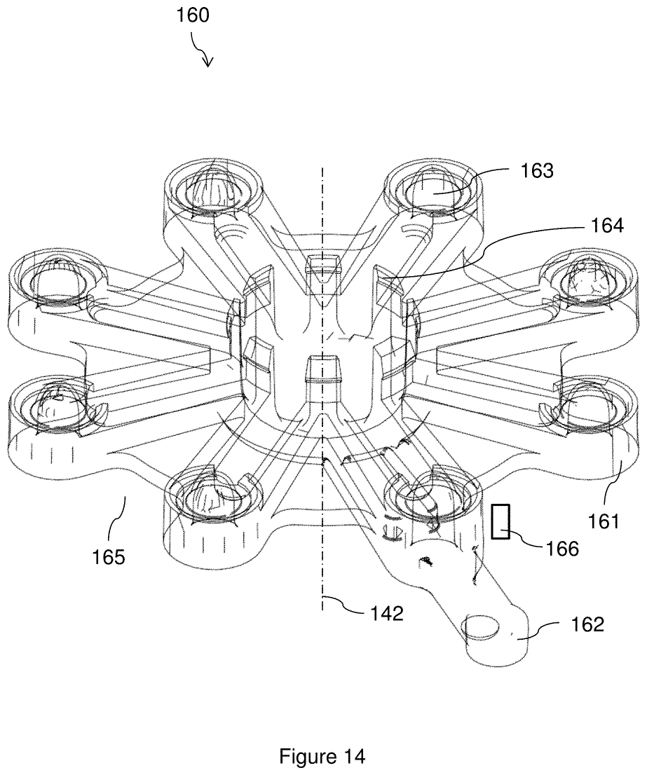

FIG. 14 represents, in perspective, an aperture seal 160 for a carousel, for example the carousel 140 illustrated in FIG. 11. The aperture seal 160 is positioned below the apertures 25, 50, 103 or 113, and the carousel 140. It has a rotatable connection 164, that is rotatably movable relative to the central axis 142 of the carousel 140. The aperture seal 160 has as many supports 161 for stoppers 163 as there are containers 100 in the carousel 140, in this case eight. A handle 162 allows the user to rotatably move the aperture seal 160 to alternately position the stoppers 163 in contact with the dispensing apertures 25, 50, 103 or 113 of the containers 100 or to move these stoppers 163 away from these apertures in such a way that each aperture of a container is facing a free gap 165 between two stoppers 163. A position sensor 166, for example a dry contact, detects the position of the aperture seal 160. For example, the position sensor 166 detects that the aperture seal 160 is in one of two positions. The stoppers 163, which here take the form of domes, are, for example, of the same material as the cap of the containers 100. The shapes (for example dome or cylinder) and each material (in particular elastomer) of the stoppers 163 are configured so that these stoppers 163 hermetically obstruct the apertures of the containers.

When introducing a carousel 140 into a liquid drop dispensing apparatus, the user has to open the seal 160, that is move the stoppers 163 away from the dispensing apertures. The signal from the sensor 166 enables the means 149 for controlling the dispensing device to control the movement of at least one point of a flexible portion of a container seal only when the stoppers are moved away from the apertures 25, 50, 103, 113 of the containers 100.

In alternatives of the embodiment illustrated in FIG. 14, the aperture seal is motorised and rotated when the dispensing device is switched on, to allow dispensing, and possibly between two of its uses, to prevent leakage or evaporation of the liquids retained in the containers 100.

Preferably, the aperture seal is removable. Rotation of the aperture seal in one direction positions the stoppers in contact with the dispensing apertures. Rotation in the other direction moves the stoppers away from the apertures.

This prevents leakage of liquid, for example, during pressure increases (due to temperature or altitude increases) or accelerations (due to shocks/vibrations or motions). This seal also prevents evaporation/degradation of liquid between uses of the device.

The stoppers of the seal are designed in such a way that their shape (dome or cylinder) and their material (in particular elastomer), hermetically obstruct the apertures of the containers.

In some embodiments, the device also comprises a position sensor for the aperture seal, the drop ejection control means being configured to control drop ejection only when the stoppers are moved away from the apertures of the containers. Drop ejection control is thus inhibited when the stopper seals the lower aperture of each container.

In embodiments, the sealing device is manually controlled by the end user by opening and closing the seal when a dedicated visual or audible signal is provided by the device. For example, a display (not represented) displays a written or symbolised instruction to manually open or close the seal.

In embodiments, the seal device is semi-automatically controlled by the end user by opening the seal when a dedicated visual or audible signal is provided by the device. The device closes the seal automatically, for example with mechanical or electromechanical means (not represented).

In embodiments, the sealing device is automatically controlled with the seal opening and closing automatically as required. For example, electromechanical means (not represented) are implemented for such automatic motion controls.

1. A device (145) for dispensing drops of liquid, comprising:
at least one container (21, 22, 100, 110) for a liquid comprising at least one internal chamber (33, 104, 118) and an upper opening (32, 102, 112), each chamber being provided with a lower aperture (25, 50, 103, 113), and with a seal (24, 120, 130) sealing the upper opening, the seal having an elastically deformable substantially planar flexible portion (123, 133),
at least one mechanical actuation means (27, 70, 146) configured to apply a deformation to a flexible portion of a seal of at least one container,
a means (147) for selecting a volume of a drop to be dispensed, from a plurality of drop volumes,
a means (148) for determining the volume of liquid present in at least one container and
a means (149) for controlling the actuation means, which controls the movement of at least one point of the flexible portion of a container seal, according to a non-constant function of the volume of the drop to be dispensed and the volume of liquid present in the container.

2. The device (145) according to claim 1, wherein the control means (149) for the actuation means (27, 70, 146) controls a speed of movement of at least one point of the flexible portion of the seal (24, 120, 130) of the container (21, 22, 100, 110).

3. The device (145) according to claim 1, wherein the control means (149) for the actuation means (27, 70, 146) controls an amplitude of movement of at least one point of the flexible portion of the seal (24, 120, 130) of the container (21, 22, 100, 110).

4. The device (145) according to claim 1, which comprises a temperature sensor (141), the control means (149) for the actuation means (27, 70, 146) controlling a pulse duration, speed and/or amplitude which, for a preselected drop volume, varies as a function of temperature.

5. The device (145) according to claim 1, wherein the actuation means (27, 146) is electromechanical and the control means (149) for the actuation means (27, 70) controls an electrical pulse duration, speed and/or amplitude of the actuation means.

6. The device (145) according to claim 1, wherein the control means (149) for the actuation means (27, 70, 146) controls a pulse duration, speed and/or amplitude which, for a preselected drop volume, is a polynomial function of degree greater than or equal to two of the volume of liquid present in the container (21, 22, 100, 110).

7. The device (145) according to claim 1, wherein the means (148) for determining the volume of liquid present in at least one container (21, 22, 100, 110) comprises a liquid volume sensor.

8. The device (145) according to claim 1, wherein the means (148) for determining the volume of liquid present in at least one container (21, 22, 100, 110) comprises a means for counting down, to the initial volume of liquid in the container, the volumes of drops already dispensed from the container.

9. The device (145) according to claim 1, which comprises a pressing sensor for the actuation means (27, 70, 146) on the flexible portion of the seal (24, 120, 130).

10. The device (145) according to claim 1, which comprises means for receiving drops of liquid dispensed by projection into the air from containers (21, 22, 100, 110) of the dispensing device.

11. The device (145) according to claim 1, wherein the seal (24, 120, 130) has an elastically deformable flexible portion (123, 133) which, for at least one plane, has a ratio of its surface area to the surface area of its projection onto said plane which is less than or equal to 1.5.

12. The device (145) according to claim 1, which comprises a carousel (140) including a structure for receiving and supporting a plurality of containers (21, 22, 100, 110).

13. The device (145) according to claim 12, wherein the carousel comprises mechanical elements configured to compensate for mechanical tolerances of the containers (21, 22, 100, 110).

14. The device (145) according to claim 1, which further comprises a seal (160) for apertures (25, 50, 103, 113) positioned below the apertures and having a rotatable connection (164), and including as many supports (161) for stoppers (163) as there are containers (21, 22, 100, 110), rotation in one direction of the aperture seal positioning the stoppers in contact with the dispensing apertures, rotation in the other direction moving these stoppers away from these apertures.

15. The device (145) according to claim 13, which comprises a position sensor for the aperture seal (160), the drop ejection control means (149) being configured to control drop ejection only when the stoppers (163) are moved away from the apertures (25, 50, 103, 113) of the containers.

16. The device (145) according to claim 13, wherein the aperture seal (160) is manually controlled by the user, by opening and closing the seal when a dedicated visual or audible signal is provided by the device.

17. The device (145) according to claim 13, wherein the aperture seal (160) is semi-automatically controlled by the end user, by opening the seal when a dedicated visual or audible signal is provided by the device, the device closing the seal automatically.

18. The device (145) according to claim 13, wherein the seal (160) is automatically controlled, the seal opening and closing automatically.

19. A method (150) for dispensing drops of liquid from at least one container (21, 22, 100, 110) for a liquid including at least one internal chamber (33, 104, 118) and an upper opening (32, 102, 112), each chamber being provided with a lower aperture (25, 50, 103, 113), and with a seal (24, 120, 130) sealing the upper opening, the seal having a substantially planar, elastically deformable flexible portion (123, 133), the method being characterised in that it comprises:
a step (153) of selecting a volume of a drop to be dispensed, from a plurality of drop volumes,
a step (155) of determining the volume of liquid present in at least one container and
a step (156, 157) of controlling at least one mechanical actuation means (27, 70, 146) configured to apply a deformation to a flexible portion of a seal of the at least one container, the movement of at least one point of the flexible portion of a container seal, according to a non-constant function of the volume of the drop to be dispensed and the volume of liquid present in the container.
